(19) United States
(12) Patent Application Publication
(10) Pub. No.: US 2023/0050858 A1
(43) Pub. Date: Feb. 16, 2023

(54) METHODS AND COMPOUNDS FOR NATURAL ORGANIC INTENSIVE SKIN REPAIR AND HEALING

(71) Applicant: Desert Harvest, Inc., Hillsborough, NC (US)

(72) Inventor: Heather Florio, Hillsborough, NC (US)

(21) Appl. No.: 17/392,975

(22) Filed: Aug. 3, 2021

(51) Int. Cl. A61K 8/9789; A61K 8/34; A61K 8/19; A61K 8/67; A61K 8/42; A61K 8/9794; A61K 8/35; A61K 8/73; A61K 8/60; A61Q 19/00; A61K 8/92

(52) U.S. Cl. CPC A61K 8/9789; A61K 8/345; A61K 8/19; A61K 8/676; A61K 8/42; A61K 8/9794; A61K 8/355; A61K 8/735; A61K 8/60; A61Q 19/00; A61K 8/922; A61K 2800/524

(57) ABSTRACT

The invention includes a skin treatment compound containing aloe vera and methods of use thereof. In one embodiment, the skin treatment compound includes aloe vera, and preferably 30×, 100× or 200× concentrated aloe vera powder, water, a Carbomer such as Ultrez powder, glycerin, lubricating ingredients, sodium hydroxide, Vitamin C, a complex mixture of skin-healing botanicals, Japanese Green Tea Extract, at least one amino acid precursor, a ceramide compound, an antioxidant, a glycosaminoglycan, and a natural preservative, preferably selected from the group consisting of lime oil, orange oil, and bergamot oil.

### TECHNICAL FIELD

The embodiments described herein include compounds and methods of application for relief from irritation and pain from cancer radiation treatments, shingles, tattoos, neuropathy, first and second degree burns, and many other ailments.

### BACKGROUND

Skin irritation in individuals is common, especially those undergoing cancer radiation treatments, as well as dealing with burns or other maladies. Relieving the pain and speeding the healing of such irritations is important to individuals. For health reasons, such individuals many times desire a natural organic solution containing no synthetic compounds or pain-relivers for aiding in the healing of such skin irritations. Moreover, having a fully natural compound for skin-care has certain advantage. For example, the skin treatment compound described in U.S. Pat. No. 9,370,545 contains synthetic lidocaine. While this synthetic composition can specifically act as an anesthetic and numb pain localized to the skin, it limits the scope of the products use. For example, by removing any pain sensation a user my subsequently touch or have the affected are run against a surface further aggravating the skin condition. Moreover, excessive use of lidocaine can have severe side-effects, such as dizziness, blurred visions, tinnitus, muscle seizures and even death. Finally, use of lidocaine is not recommended for areas of the body such as around the eyes or mouth, and can cross-react with other drugs.

### SUMMARY OF THE INVENTION

Embodiments of the compounds and methods described herein aid in skin repair and relieve irritation and pain that may result from various treatments including radiation, shingles, tattoos and tattoo removal, and neuropathy, among other ailments.

In one embodiment, a method for making a skin treatment compound includes making a Phase A portion of the skin treatment compound including: mixing Ultrez powder with water to wet the Ultrez powder; adding glycerin; and heating and holding the Phase A portion at 170° F. Ultrez polymer is a hydrophobically modified cross-linked acrylate copolymer (Acrylates/CI0-30 Alkyl Acrylate Crosspolymer). The method further includes making a Phase B portion of the skin treatment compound including: adding lubricants; heating the Phase B portion at 170° F.; stirring the Phase B portion; and resting the Phase B portion for ten minutes. The method further includes mixing the Phase B portion with the Phase A portion. The method further includes adding sodium hydroxide to the Phase B portion with the Phase A portion to form a Phase C portion. The method further includes making a Phase E portion of the skin treatment compound including: dissolving

Vitamin C in water; adding a Phase D portion with Japanese Green Tea Extract, Aloe Vera Powder, and natural preservatives; and adding the Phase C portion.

The method further includes mixing the resulting skin treatment compound. Optionally, the lubricants include at least one ingredient selected from a list consisting of Glyceryl Stearate, Stearic Acid, Cetyl Alcohol, Hi Oleic Safflower Oil, Rosehip Seed Oil, Vitamin E, and Chia Seed Oil. Alternatively, the lubricants include Glyceryl Stearate, Glyceryl Undecylenate, Glyceryl Caprylate, Stearic Acid, Cetyl Alcohol, Hi Oleic Safflower Oil, Rosehip Seed Oil, Vitamin E, and Chia Seed Oil, Simmondsia Chinensis (Jojoba) Seed Oil.

Optionally, the Phase D portion includes at least one botanical selected from a list consisting of Chamomilla Recutita (Matricaria) Extract, Calendula Officinalis (Calendula) Flower Extract, Anthemis Nobilis (Roman Chamomile) Flower Extract, Centaurea Cyanus (Cornflower) Flower Extract, Tilia Cordata (Linden) Flower Extract, Vitis Vinifera (Grapeseed) Extract, Tabebuia Impetiginosa (Pau d′ Arco) Extract, and Thymus Vulgaris (Thyme) Oil Extract. Alternatively, the Phase D portion includes Chamomilla Recutita (Matricaria) Extract, Calendula Officinalis (Calendula) Flower Extract, Anthemis Nobilis (Roman Chamomile) Flower Extract, Centaurea Cyanus (Cornflower) Flower Extract, Tilia Cordata (Linden) Flower Extract, Vitis Vinifera (Grapeseed) Extract, Tabebuia Impetiginosa (Pau d′ Arco) Extract, and Thymus Vulgaris (Thyme) Oil Extract. Optionally, the natural preservatives include Leuconostoc/Radish Root Ferment Filtrate, Lonicera Japonica (Honeysuckle) Flower Extract, Lonicera Caprifolium (Honeysuckle) Extract, Populus Tremuloides (Aspen) Bark Extract, Gluconolactone, Orange Oil extract, Lime Oil Extract, and Bergamot (Citrus bergamia) extract.

In one alternative embodiment, a skin treatment compound includes one or more amino acid precursors, such as L-Threonine which is a precursor to the amino acids glycine and serine and promotes the formation of collagen and elastin.

In one alternative embodiment, a skin treatment compound includes one or more ceramides, such as ceramide NG, is an intercellular lipid naturally found in skin that bonds dead skin cells together to create a water barrier to prevent dehydration.

In one alternative embodiment, a skin treatment compound includes one or more antioxidants, such as Coenzyme Q10 (CoQ10) which is a kind of fat-soluble quinone compound that acts as an antioxidant and can reduce cellular damage due to injury, UV radiation exposure and the like.

In one alternative embodiment, a skin treatment compound includes one or more glycosaminoglycans, such as hyaluronic acid, which is a complex sugar molecule that is produce in the skin and joints and helps collage bind to water allowing the skin to retain moisture.

In one alternative, the method further includes adjusting a pH of the skin treatment compound to between 6.8 and 7.2. In one alternative, the method further includes adjusting a viscosity of the skin treatment compound to between 35,000 and 45,000 centipoise (cps) at 25° C.

In one embodiment, a skin treatment compound includes water; Ultrez powder; glycerin; skin healing botanicals; sodium hydroxide; Vitamin C; a Phase D portion; Japanese Green Tea Extract; Aloe Vera, and preferably 30×, 100× OR 200× concentrated Aloe Vera Powder; and natural preservatives. Optionally, the lubricating ingredients include at least one ingredient selected from a list consisting of Glyceryl Stearate, Stearic Acid, Cetyl Alcohol, Hi Oleic Safflower Oil, Rosehip Seed Oil, Vitamin E, and Chia Seed Oil. Alternatively, the lubricating ingredients include Glyceryl Stearate, Glyceryl Caprylate, Glyceryl Undecylenate, Stearic Acid, Cetyl Alcohol, Hi Oleic Safflower Oil, Rosehip Seed Oil, Vitamin E, and Chia Seed Oil.

In one alternative, the Phase D portion includes at least one botanical selected from a list consisting of Chamomilla Recutita (Matricaria) Extract, Calendula Officinalis (Calendula) Flower Extract, Anthemis Nobilis (Roman Chamomile) Flower Extract, Centaurea Cyanus (Cornflower) Flower Extract, Tilia Cordata (Linden) Flower Extract, Vitis Vinifera (Grapeseed) Extract, Tabebuia Impetiginosa (Pau d′ Arco) Extract, and Thymus Vulgaris (Thyme) Oil Extract. Optionally, the Phase D portion includes Chamomilla Recutita (Matricaria) Extract, Calendula Officinalis (Calendula) Flower Extract. Anthemis Nobilis (Roman Chamomile) Flower Extract, Centaurea Cyanus (Cornflower) Flower Extract, Tilia Cordata (Linden) Flower Extract, Vitis Vinifera (Grapeseed) Extract, Tabebuia Impetiginosa (Pau d′ Arco) Extract, and Thymus Vulgaris (Thyme) Oil Extract. Alternatively, the natural preservatives include Leuconostoc/Radish Root Ferment Filtrate, Lonicera Japonica (Honeysuckle) Flower Extract, Lonicera Caprifolium (Honeysuckle) Extract, Populus Tremuloides (Aspen) Bark Extract, Gluconolactone Orange (Citrus sinensis) Oil extract, Lime (Citrus aurantiifolia) Oil Extract, and Bergamot (Citrus bergamia) extract.

Optionally, the natural preservatives include at least one botanical selected from a list consisting of Leuconostoc/Radish Root Ferment Filtrate, Lonicera Japonica (Honeysuckle) Flower Extract, Lonicera Caprifolium (Honeysuckle) Extract, Populus Tremuloides (Aspen) Bark Extract, and Gluconolactone.

Optionally, a pH of the skin treatment compound is between 6.8 and 7.2. Alternatively, a viscosity of the skin treatment compound is between 35,000 and 45,000 centipoise (cps) at 25° C.

In another embodiment, a skin treatment compound includes aloe vera. The aloe vera is Barbadensis Miller species. Optionally, the skin treatment compound includes Calendula. Alternatively, the skin treatment compound includes natural preservatives. Optionally, the natural preservatives include Leuconostoc/Radish Root Ferment Filtrate, Lonicera Japonica (Honeysuckle) Flower Extract, Lonicera Caprifolium (Honeysuckle) Extract, Populus Tremuloides (Aspen) Bark Extract, and Gluconolactone. Alternatively, the natural preservatives include at least one botanical selected from a list consisting of Leuconostoc/Radish Root Ferment Filtrate, Lonicera Japonica (Honeysuckle) Flower Extract, Lonicera Caprifolium (Honeysuckle) Extract, Populus Tremuloides (Aspen) Bark Extract, and Gluconolactone. The skin treatment compound may further include includes one or more of the following: L-Threonine, ceramide NG, coenzyme Q10 (CoQ10); hyaluronic acid, orange oil, lime oil, and bergamot oil.

In another embodiment, a method of treating skin irritation includes treating the skin irritation with an effective amount of a compound. The compound includes aloe vera. The aloe vera is Barbadensis Miller species. Optionally, the skin treatment compound includes Calendula. Alternatively, the skin treatment compound includes natural preservatives. Optionally, the natural preservatives include Leuconostoc/Radish Root Ferment Filtrate, Lonicera Japonica (Honeysuckle) Flower Extract, Lonicera Caprifolium (Honeysuckle) Extract, Populus Tremuloides (Aspen) Bark Extract, and Gluconolactone Ferment Filtrate, Lonicera Japonica (Honeysuckle) Flower Extract, Lonicera Caprifolium (Honeysuckle) Extract, Populus Tremuloides (Aspen) Bark Extract, and Gluconolactone.

The skin treatment compound may further include includes one or more of the following: L-Threonine, ceramide NG, coenzyme Q10 (CoQ10), hyaluronic acid, orange oil, lime oil, and bergamot oil.

The invention includes a skin treatment compound, and methods of making the same containing aloe vera and methods of use thereof. In one embodiment, the skin treatment compound includes aloe vera, and preferably 30×, 100× or 200× concentrated aloe vera powder, water, a Carbomer such as Ultrez powder, glycerin, lubricating ingredients, sodium hydroxide, Vitamin C, a complex mixture of skin-healing botanicals, Japanese Green Tea Extract, at least one amino acid precursor, a ceramide compound, an antioxidant, a glycosaminoglycan, and a natural preservative, preferably selected from the group consisting of lime oil, orange oil, and bergamot oil.

In another embodiment, a skin treatment compound may include one or more of the following: aloe vera, and preferably 30×, 100× or 200× concentrated aloe vera powder; water; Ultrez powder; glycerin; lubricating ingredients; sodium hydroxide; Vitamin C; a Phase D portion of skin-healing botanicals; Japanese Green Tea Extract; at least one amino acid precursor; a ceramide compound; an antioxidant; a glycosaminoglycan; and a natural preservative selected from the group consisting of lime oil, orange oil, and bergamot oil.

In another embodiment, a skin treatment compound may include one or more of the following: aloe vera, and preferably 30×, 100× or 200× concentrated aloe vera powder; water; Ultrez powder; glycerin; lubricating ingredients; sodium hydroxide; Vitamin C; a Phase D portion of skin-healing botanicals; Japanese Green Tea Extract; L-Threonine; ceramide NG; coenzyme Q10 (CoQ10); hyaluronic acid; and a natural preservative selected from the group consisting of lime oil, orange oil, and bergamot oil.

### BRIEF DESCRIPTION OF THE FIGURES

FIG. 1 shows an exemplary method of making Phase A of a compound for intensive skin repair and healing;

FIG. 2 shows an exemplary method of making Phase B of a compound for intensive skin repair and healing;

FIG. 3 shows an exemplary method of making Phase C of a compound for intensive skin repair and healing;

FIG. 4 shows an exemplary method of making Phase E and Phase F of a compound for intensive skin repair and healing.

### DETAILED DESCRIPTION OF THE INVENTION

Embodiments of methods and compounds are described for skin repair and healing and relief from irritation and pain that may result from various treatments including radiation, shingles, tattoos and tattoo removal, neuropathy, or other skin-related issues, and are for illustrative purposes only. The term the “compound” is used repeatedly herein. The term “compound” generally refers to the various embodiments described herein that include in many embodiments a combination of ingredients including aloe vera (in a preferred embodiment Barbadensis Miller species), and Calendula, an amino acid precursor, a ceramide compound, an antioxidant, a glycosaminoglycan, as well as a combination of many other ingredients, and the scope of the coverage of the claims is only intended to be limited by the specifics of the claims and not the examples described in relation to possible compounds. The compound typically is delivered in a cream or lotion form, but alternative liquids or gels may be created. The following provides further description of certain embodiments of the methods and compounds. As described and claimed here, certain terms are defined and used interchangeably.

As used herein, the term “period of time” or “duration of time” means more than a single dosing, application, or administration of the compound. More specifically, these terms mean the compound is administered/applied one or more times per day over a period of seven or more days, wherein generally no two consecutive days pass without the application of the compound and the individual applies the compound at least three or four days in any seven- day period, more preferably four or more days in any seven-day period, more preferably five or more days in any seven-day period, more preferably six or more days in any seven-day period, and most preferably seven consecutive days in any seven-day period. For example, the individual can apply the compound every day, wherein the compound is applied multiple times over the course of the day or the individual may apply the compound a single time in a day.

As used herein, the term “effective amount” or “amount effective to” refers to an amount of the compound required to increase the healing of skin and reduce pain or irritation of the skin. It will be understood by those skilled in the art that a one time, single application of embodiments of the compound may not be effective. Furthermore, it will be understood by those skilled in the art that administering a single dose followed with multiple consecutive days of non-dosing or non-supplementation will not achieve the effective amount as described.

This disclosure provides an important understanding of how the compound may provide healing and relief from pain and irritation when an effective amount is applied to affected areas over a period of time.

In many embodiments, the compound includes ingredients of aloe vera. In some alternatives, the specific species of Barbadensis Miller is used for the aloe vera. In many alternatives, Calendula is used as an ingredient as well. The addition of Calendula is thought to be unique, as used in the combination of ingredients in the compound, as it has been studied in relation to radiation burns specifically. Each of the botanicals in the combination of ingredients has a property for skin health and healing. Note that the combination of botanicals provided herein, as well as the percentages of those botanicals, is merely exemplary; and the exact percentages and botanicals used may change and still be within the scope of the compound.

In many embodiments, the compound includes ingredients of aloe vera. In some alternatives, the specific species of Barbadensis Miller is used for the aloe vera. In many alternatives, at least one amino acid precursor, such as L-threonine is used as an ingredient as well.

In many embodiments, the compound includes ingredients of aloe vera. In some alternatives, the specific species of Barbadensis Miller is used for the aloe vera. In many alternatives, a ceramide compound, such as ceramide NG is used as an ingredient as well.

In many embodiments, the compound includes ingredients of aloe vera. In some alternatives, the specific species of Barbadensis Miller is used for the aloe vera. In many alternatives, an antioxidant, such as CoQ10 is used as an ingredient as well.

In many embodiments, the compound includes ingredients of aloe vera. In some alternatives, the specific species of Barbadensis Miller is used for the aloe vera. In many alternatives, a glycosaminoglycan, such as hyaluronic acid is used as an ingredient as well.

In many alternatives, another unique feature of the compound is that the compound includes a combination of botanicals that serve as a natural preservative. By using the unique combination of preservatives, parabens or other unnatural chemicals are not needed. Furthermore, the combination of botanical preservatives is organic, in the sense of typical FDA standards. This allows the compound to be considered entirely 100% organic.

In some alternatives, the aloe vera is organic aloe Barbadensis Miller (research has shown that it penetrates to the cellular level and carries the other ingredients deeper into the tissues). Research using aloe vera on radiation burns has been conducted since the 1930s. According to dermatological research, aloe vera concentrate may help reduce the incidence of skin breakdown, which is associated with repeated radiation treatments. This makes the compound particularly effective at dealing with radiation burns and other radiation-related skin degradation. The compound also may be particularly effective for first-aid kit manufacturers for restaurant and industrial burns, as well as dermatologists for use following dermabrasion treatments.

In one embodiment, the compound additionally includes one or more ingredients from the following list:
Glyceryl Stearate (Natural Skin Lubricant)
Glyceryl Caprylate (Natural Skin Lubricant)
Glyceryl Undecylenate (Natural Skin Lubricant)
Stearic Acid (Plant-based Fatty Acid)
Carthamus Tinctorius (Safflower) Seed Oil
Rosa Canina (Rosehips) Fruit Oil
Sodium Ascorbyl Phosphate (Vitamin C)
Salvia Sinensis (Chia) Seed Oil
Vegetable Glycerin
Cetyl Alcohol (from Coconut Oil)
Carbomer (Natural Thickener)
Calendula Officinalis (Pot Marigold) Flower Extract
Chamomilla Recutita (Chamomile) Flower Extract
Matricaria Chamomilla (Roman Chamomile) Flower Extract
Tilia Officinalis (Linden) Extract
Hypericum Perforatum (St. John's Wort) Extract
Centaurea Cyanus (Cornflower) Extract
Vitis Vinifera (Grape) Seed Extract
Tabebuia Impetiginosa (Pau d′ Arco) Extract
Camellia Sinensis (Japanese Green Tea) Leaf Extract
Tocopheryl Acetate (Vitamin E)
at least one Amino Acid Precursor
a Ceramide Compound
an Antioxidant
a Glycosaminoglycan
Xanthan Gum
Acrylates/C10-30 Alkyl Acrylate Crosspolymer
Copper Tripeptide-1
Echinacea Purpurea (Coneflower) Extract

In many embodiments, a natural preservative is used. This natural preservative includes Leuconostoc/Radish Root Ferment Filtrate, which functions as a natural preservative. The natural preservative further includes Lonicera Japonica (Honeysuckle) Flower Extract, Lonicera Caprifolium (Honeysuckle) Extract, and Populus Tremuloides (Aspen) Bark Extract. The natural preservative also includes Gluconolactone, which functions as a natural skin conditioner. In additional embodiments, a natural preservative may include lime oil, orange oil, or bergamot oil, or a combination of the same.

Various botanicals have been selected for inclusion in the unique compound. Chamomilla Recutita (Matricaria) extract is used because it may function as a healing balm to treat burns, wounds, eczema, rashes, anal irritation, and sunburn. It has overall healing and soothing effects.

Calendula Officinalis (Calendula) flower extract is used because it may function as a soothing anti-inflammatory agent (high content of polysaccharides and terpenoids) and it has antiseptic properties. The resulting compound may be used to treat eczema, burns, wounds, and other types of skin irritations. There are clinical studies that demonstrate that calendula can partially prevent some forms of acute dermatitis related to radiation therapy in breast cancer patients. It has been shown to enhance collagen production and aid in preventing excessive scar development. It has a demonstrated ability to increase formation of new blood vessels. Bruises disperse much more quickly with application of this botanical. Last but not least, calendula is an immune stimulant.

Anthemis Nobilis (Roman Chamomile) flower extract is used because it may function similarly to Matricaria in its properties. It is a soothing and calming anti-inflammatory botanical.

Because of its gentle action, Centaurea Cyanus (cornflower) flower extract is commonly used around the eyes for its antiseptic properties. Its soothing qualities and emolliency aids in treating aging skin.

Tilia Cordata (Linden) flower extract is used because it may have a positive effect on reducing cellulitis and edema. It has been shown to decrease dry skin as well as decrease the dark spots frequently seen on aging skin.

Hypericum Perforatum (St. John's Wort) extract is used because of its history of treating bruises, burns, sores, and sprains.

Neuralgia is used because it may have pain relieving activity related to flavonoid content. It has been shown to be active in preventing skin infections. It has strong antiviral activity, decreasing the appearance of cold sores and fever blisters.

Vitis Vinifera (Grapeseed) extract is used because it contains high levels of flavonoids and procyanidins. It is a very strong antioxidant and free radical destroyer. It has been shown to strengthen blood vessels and increase circulation with a high content of resveratrol contributing to overall vascular health as well as immune system support. Tabebuia Impetiginosa (Pau d′ Arco) extract is used because it may have both anti-fungal and anti-viral properties. It has been used to treat eczema and other forms of dermatitis. It also has been shown to reduce pain and inflammation. It has been reputed to have anti-cancer effects, although the required dosage for this property has side effects.

Thymus Vulgaris (Thyme) extract is used because it may have antiseptic, antiviral, and antifungal properties. It has been shown to be effective for bruises and wounds. It has been used to treat cases of crabs and lice, as well as scabies. It is a strong antioxidant.

L-Threonine which is a precursor to the amino acids glycine and serine and is used as it promotes the formation of collagen and elastin and promote skin healing and pain relief.

ceramide NG is used as it is is an intercellular lipid naturally found in skin that bonds dead skin cells together to create a water barrier to prevent dehydration and promote skin healing and pain relief.

Coenzyme Q10 (CoQ10) is used as it is a fat-soluble quinone compound that acts as an antioxidant and can reduce cellular damage due to injury, UV radiation exposure and the like.

Hyaluronic acid is a complex sugar molecule and is used to help collage bind to water allowing the skin to retain moisture and promote healing and pain relief.

While there are certain similarities between many of the properties of these botanicals, there is believed to be interaction and possible synergy among them with regard to their remedial potential.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

In one embodiment, the compound may be made according to the following process. The percentage of the total composition is shown in the first column and the named ingredient is listed in the second column. The preparation of the compound is listed as including Phases A-F.

| PHASE A | INCI NAME |
|---|---|
| 10.01-50.00% Deionized Water | Water |
| .1-.2% Ultrez 10 | Carbomer |
| 1.00-2.50% Glycerin 99% | Glycerin |

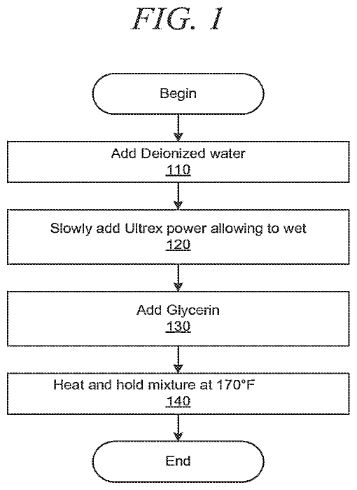

FIG. 1 shows an exemplary method of preparing Phase A of the compound. In step 110, in a mixing tank large enough for the entire batch, deionized water is added. In step 120, slowly add the Ultrez 10 powder and allow it to wet. In step 130, once the Ultrez 10 powder is wetted, add the glycerin and mix slowly until the Ultrez 10 is totally dispersed. In step 140, heat and hold the mixture at 170° F., while preparing Phase B. Cover tank to avoid as much water loss as possible.

| PHASE B | INCI NAME |
|---|---|
| 2.51-10.00% Glyceryl Stearate | Glyceryl Stearate |
| 2.51-10.00% Stearic Acid | Stearic Acid |
| 2.51-10.00% Cetyl Alcohol | Cetyl Alcohol |
| 2.51-10.00% Hi Oleic Safflower Oil | Carthamus Tinctorius (Safflower) Seed |
| | Oil |
| 1.00-2.50% Rosehip Seed Oil | Rosa Canina (Rosehip) Fruit Oil |
| 0.10-0.99% Vitamin E | Tocopheryl Acetate |
| 1.00-2.50% Chia Seed Oil | Salvia Sinensis (Chia) Seed Oil |

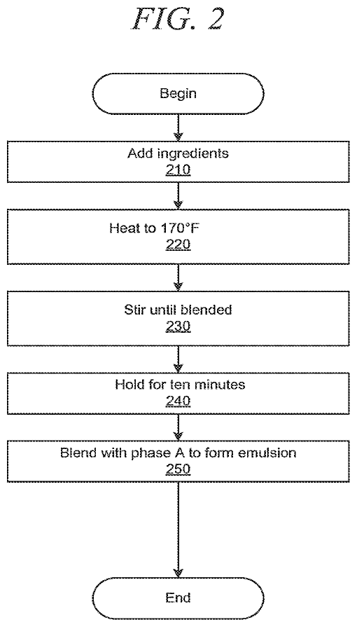

FIG. 2 shows an exemplary method of preparing Phase B. In step 210, in a tank large enough for Phase B, add the Phase B ingredients. In step 220, the mixture is heated to 170° F. In step 230, the mixture is stirred and the temperature is held at 170° F. until all ingredients have melted and blended together. In step 240, the mixture is allowed to sit at temperature for an additional ten minutes. In step 250, the mixture is slowly added to Phase A with continued mixing until a smooth, well-blended emulsion has formed and the heat is turned off.

| PHASE C | INCI NAME |
|---|---|
| 0.10-0.99% Sodium Hydroxide | Sodium Hydroxide |
| (10% aqueous solution) | |

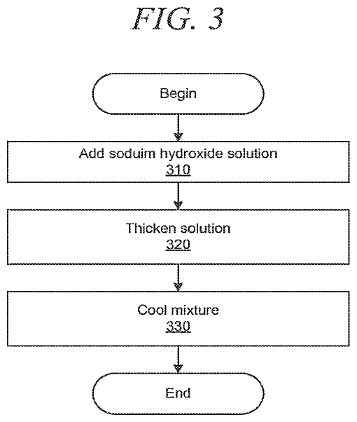

FIG. 3 shows an exemplary method of preparing Phase C. In step 310, immediately the sodium hydroxide solution is added to the Phase A and Phase B, while stirring. In step 320, the emulsion should begin to thicken somewhat. Sodium hydroxide solutions are considerably exothermic and are most easily prepared before the rest of the mix is begun. In step 330, the mixtures then is set aside to cool to room temperature and covered until needed. 10% is w/w in this instance. The emulsion must reach 104° F. before the addition of Phase E.

| PHASE D | Botanical Compound |
|---|---|
| 1.00-2.50% Chamomilla Recutita Extract | (Matricaria) Extract |
| 0.10-0.99% Calendula Officinalis Flower | (Calendula) Flower Extract |
| Extract | |
| <0.09% Anthemis Nobilis Flower Extract | (Roman Chamomile) Flower Extract |
| 0.10-0.99% Echinacea Purpurea Flower | (Coneflower) Flower Extract |
| Extract | |
| 0.10-0.99% Tilia Cordata Flower Extract | (Linden) Flower Extract |
| 0.10-0.99% Vitis Vinifera Extract | (Grapeseed) Extract |
| 0.10-0.99% Tabebuia Impetiginosa Extract | (Pau d' Arco) Extract |

In an exemplary embodiment, Phase D includes the above ingredients. To make Phase D, the extracts and mix are combined until an even dispersion is achieved. The combination will make up 1.00% or more of the entire formula.

| PHASE E | NAME |
|---|---|
| 0.10-0.99% Japanese Green Tea Extract | Camellia Sinensis Extract |
| 1.00-2.50% Vitamin C (soluble derivative) | Sodium Ascorbyl Phosphate |
| 10.01-50.00% Organic 30x, 100x or 200X Aloe Vera | Aloe Barbadensis Inner Leaf Juice |
| Powder | |
| 10.01-50.00% Deionized Water | Water |
| .0.10-0.99% at least one amino acid precursor | L-Threonine |
| 0.10-0.99% a ceramide compound | Ceramide NG |
| 0.10-0.99% an antioxidant | CoQ10 |
| 0.10-0.99% a glycosaminoglycan | Hyaluronic Acid |
| 0.10-0.99% of one or more Natural Preservatives | Leuconostoc/Radish Root Ferment |
| | Filtrate, Lonicera Japonica |
| | (Honeysuckle) Flower Extract, |
| | Lonicera Caprifolium (Honeysuckle) |
| | Extract, Populus Tremuloides (Aspen) |
| | Bark Extract, Simmondsia Chinensis |
| | (Jojoba) Seed Oil, Gluconolactone, |
| | lime oil, orange oil, and bergamot oil |

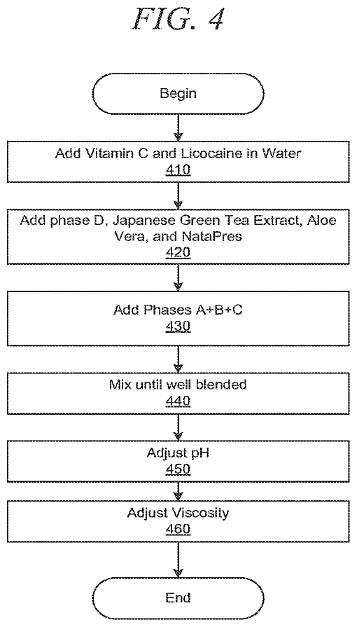

While Phases A+B+C are cooling, Phase E is prepared. FIG. 4 shows an exemplary embodiment of the process of making Phase E and completing the mixture in Phase F. In step 410, in a separate container large enough for Phase E, Vitamin C are dissolved in the deionized water. In step 420, after the ingredients are dissolved, the Phase D mixture, Japanese Green Tea Extract, Aloe Vera Powder, L-Threonine, Ceramide NG, CoQ10, Hyaluronic Acid and Natural Preservatives are added. In step 430, the mixture is mixed well until evenly blended. In step 430, once Phases A+B+C have reached a temperature of 104° F., Phase E is slowly added to the A+B+C mixture in the tank. In step 440, the mixture is mixed well for ten minutes or until well blended.

Phase F includes quality control procedures for ensuring that the resulting compound is complete. Note that the compound typically is applied in lotion or cream form.

In step 450, the pH of the compound is adjusted. To do this, a sample is collected from the finished mixture and taken to the quality control lab for inspection. The pH should be between 6.8 and 7.2. At times, homogenization of the compound may be required. In step 460, the viscosity of the mixture is performed as needed. The viscosity at 25° C. should be between 35,000 and 45,000 centipoise (cps). Timings required for mixing are dependent on the tank, batch size, type of mixer, and speed of mixer.

Additional formulations of the invention may include Copper Tripeptide-1, Acrylates/C10-30 Alkyl Acrylate Crosspolymer, Xanthan Gum, Glyceryl Caprylate, and Glyceryl Undecylenate. In a preferred embodiment, one or more of the following ingredients may be include 0.10-0.99% contribution to the overall composition.

Although the foregoing description is directed to certain embodiments, it is noted that other variations and modifications will be apparent to those skilled in the art and may be made without departing from the spirit or scope of the disclosure. As used herein the term INCI means the “International Nomenclature Cosmetic Ingredient.” Moreover, features described in connection with one embodiment may be used in conjunction with other embodiments, even if not explicitly stated above.

1. A skin treatment compound comprising:
water;
Ultrez powder;
glycerin;
lubricating ingredients;
sodium hydroxide;
Vitamin C;
a Phase D portion of skin-healing botanicals;
Japanese Green Tea Extract;
at least one amino acid precursor;
a ceramide compound;
an antioxidant;
a glycosaminoglycan;
Aloe Vera Powder; and
a natural preservative selected from the group consisting of lime oil, orange oil, and bergamot oil.

2. The compound of claim 1, wherein the lubricating ingredients include at least one botanical selected from a list consisting of Glyceryl Stearate, Glyceryl Undecylenate, Glyceryl Caprylate Stearic Acid, Cetyl Alcohol, Hi Oleic Safflower Oil, Rosehip Seed Oil, Vitamin E, Simmondsia Chinensis (Jojoba) Seed Oil, and Chia Seed Oil.

3. The compound of claim 1, wherein the lubricating ingredients include Glyceryl Stearate, Glyceryl Undecylenate, Glyceryl Caprylate, Stearic Acid, Cetyl Alcohol, Hi Oleic Safflower Oil, Rosehip Seed Oil, Vitamin E, Simmondsia Chinensis (Jojoba) Seed Oil, and Chia Seed Oil.

4. The compound of claim 1, wherein the Phase D portion includes at least one botanical selected from a list consisting of Chamomilla Recutita (Matricaria) Extract, Calendula Officinalis (Calendula) Flower Extract, Anthemis Nobilis (Roman Chamomile) Flower Extract, Centaurea Cyanus (Cornflower) Flower Extract, Tilia Cordata (Linden) Flower Extract, Vitis Vinifera (Grapeseed) Extract, Tabebuia Impetiginosa (Pau d′ Arco) Extract, Echinacea Purpurea (Coneflower) Extract, and Thymus Vulgaris (Thyme) Oil Extract.

5. The compound of claim 1, wherein the Phase D portion includes Chamomilla Recutita (Matricaria) Extract, Calendula Officinalis (Calendula) Flower Extract. Anthemis Nobilis (Roman Chamomile) Flower Extract, Centaurea Cyanus (Cornflower) Flower Extract, Tilia Cordata (Linden) Flower Extract, Vitis Vinifera (Grapeseed) Extract, Tabebuia Impetiginosa (Pau d′ Arco) Extract, Echinacea Purpurea (Coneflower) Extract, and Thymus Vulgaris (Thyme) Oil Extract.

6. The compound of claim 1, wherein the natural preservatives further include one or more additional natural preservatives selected from the group consisting of: include Leuconostoc/Radish Root Ferment Filtrate, Lonicera Japonica (Honeysuckle) Flower Extract, Lonicera Caprifolium (Honeysuckle) Extract, Populus Tremuloides (Aspen) Bark Extract, Echinacea Purpurea (Coneflower) Extract, and Gluconolactone.

7. The compound of claim 1, wherein the natural preservatives include at least one botanical selected from a list consisting of Leuconostoc/Radish Root Ferment Filtrate, Lonicera Japonica (Honeysuckle) Flower Extract, Lonicera Caprifolium (Honeysuckle) Extract, Populus Tremuloides (Aspen) Bark Extract, Echinacea Purpurea (Coneflower) Extract, and Gluconolactone.

8. The compound of claim 1, wherein a pH of the skin treatment compound is between 6.8 and 7.2.

9. The compound of claim 1, wherein a viscosity of the skin treatment compound is between 35,000 and 45,000 centipoise (cps) at 25° C.

10. The compound of claim 1, wherein said at least one amino acid precursor comprises L-Threonine.

11. The compound of claim 1, wherein said ceramide comprises ceramide NG.

12. The compound of claim 1, wherein said antioxidant comprises coenzyme Q10 (CoQ10).

13. The compound of claim 1, wherein said glycosaminoglycan comprises hyaluronic acid.

14. A method of treating a skin, comprising applying an effective amount of the composition of claim 1 to the skin.

15. A skin treatment compound comprising:
aloe vera
Calendula;
at least one amino acid precursor;
a ceramide compound;
an antioxidant;
a glycosaminoglycan; and
one or more natural preservatives.

16. The compound of claim 11, wherein said at least one amino acid precursor comprises L-Threonine.

17. The compound of claim 11, wherein said ceramide comprises ceramide NG.

18. The compound of claim 11, wherein said antioxidant comprises coenzyme Q10 (CoQ10).

19. The compound of claim 11, wherein said glycosaminoglycan comprises hyaluronic acid.

20. The compound of claim 11, wherein the natural preservatives include one or more natural preservatives selected from the group consisting of: include Leuconostoc/Radish Root Ferment Filtrate, Lonicera Japonica (Honeysuckle) Flower Extract, Lonicera Caprifolium (Honeysuckle) Extract, Populus Tremuloides (Aspen) Bark Extract, Echinacea Purpurea (Coneflower) Extract, and Gluconolactone.

21. The compound of claim 11, wherein the natural preservatives include at least one botanical selected from a list consisting of Leuconostoc/Radish Root Ferment Filtrate, Lonicera Japonica (Honeysuckle) Flower Extract, Lonicera Caprifolium (Honeysuckle) Extract, Populus Tremuloides (Aspen) Bark Extract, Echinacea Purpurea (Coneflower) Extract, and Gluconolactone.

22. The compound of claim 11, wherein the natural preservatives include one or more natural preservatives selected from the group consisting of: lime oil, orange oil, and bergamot oil.

23. A method of treating a skin, comprising applying an effective amount of the composition of claim 11 to the skin.

24. A method for making a skin treatment compound, the method comprising:
making a Phase A portion of the skin treatment compound including:
mixing Ultrez powder with water to wet the Ultrez powder; adding glycerin; and
heating and holding the Phase A portion at 170° F.;
making a Phase B portion of the skin treatment compound including: adding Phase B lubricating ingredients;
heating the Phase B portion at 170° F.; stirring the Phase B portion;
resting the Phase B portion for 10 minutes; and mixing the Phase B portion with the Phase A portion;
making a Phase C portion;
adding sodium hydroxide to the Phase B portion with the Phase A portion to form a Phase C portion;
making a Phase D portion;
making a Phase E portion of the skin treatment compound including dissolving Vitamin C in water;
adding a Phase D portion, Japanese Green Tea Extract, Aloe Vera Powder, L-Threonine, Ceramide NG, CoQ10, Hyaluronic Acid, and natural preservatives;
adding the Phase C portion; and
mixing the resulting skin treatment compound.

25. The method of claim 1, wherein the lubricating ingredients include at least one botanical selected from a list consisting of Glyceryl Stearate, Glyceryl Undecylenate, Glyceryl Caprylate, Stearic Acid, Cetyl Alcohol, Hi Oleic Safflower Oil, Rosehip Seed Oil, Vitamin E, Simmondsia Chinensis (Jojoba) Seed Oil, and Chia Seed Oil.

26. The method of claim 1, wherein the lubricating ingredients include Glyceryl Stearate, Glyceryl Undecylenate, Glyceryl Caprylate, Stearic Acid, Cetyl Alcohol, Hi Oleic Safflower Oil, Rosehip Seed Oil, Vitamin E, Simmondsia Chinensis (Jojoba) Seed Oil, and Chia Seed Oil.

27. The method of claim 1, wherein the Phase D portion includes at least one botanical selected from a list consisting of Chamomilla Recutita (Matricaria) Extract, Calendula Officinalis (Calendula) Flower Extract, Anthemis Nobilis (Roman Chamomile) Flower Extract, Centaurea Cyanus (Cornflower) Flower Extract, Tilia Cordata (Linden) Flower Extract, Vitis Vinifera (Grapeseed) Extract, Tabebuia Impetiginosa (Pau d′ Arco) Extract, Echinacea Purpurea (Coneflower) Extract, and Thymus Vulgaris (Thyme) Oil Extract.

28. The method of claim 1, wherein the Phase D portion includes Chamomilla Recutita (Matricaria) Extract, Calendula Officinalis (Calendula) Flower Extract, Anthemis Nobilis (Roman Chamomile) Flower Extract, Centaurea Cyanus (Cornflower) Flower Extract, Tilia Cordata (Linden) Flower Extract, Vitis Vinifera (Grapeseed) Extract, Tabebuia Impetiginosa (Pau d′ Arco) Extract, Echinacea Purpurea (Coneflower) Extract, and Thymus Vulgaris (Thyme) Oil Extract.

29. The method of claim 1, wherein the natural preservatives include Leuconostoc/Radish Root Ferment Filtrate, Lonicera Japonica (Honeysuckle) Flower Extract, Lonicera Caprifolium (Honeysuckle) Extract, Populus Tremuloides (Aspen) Bark Extract, Echinacea Purpurea (Coneflower) Extract, Gluconolactone, lime oil, orange oil, and bergamot oil.

30. The method of claim 1, further comprising adjusting a pH of the skin treatment compound to between 6.8 and 7.2.

31. The method of claim 1, further comprising adjusting a viscosity of the skin treatment compound to between 35,000 and 45,000 centipoise (cps) at 25° C.

32. A method of treating a skin, comprising applying an effective amount of the composition generated by the method of claim 21 to the skin.
